(19) United States
(12) Patent Application Publication
(10) Pub. No.: US 2004/0002086 A1
(43) Pub. Date: Jan. 1, 2004

(54) Monoclonal antibody for Nkx3.1 and method for detecting the same

(75) Inventors: Cory Abate-Shen, Warren, NJ (US); Michael M. Shen, Warren, NJ (US); Minjung Kim, Piscataway, NJ (US)

Correspondence Address: Richard R. Muccino, Esq., 758 Springfield Avenue, Summit, NJ, 07901, US

(73) Assignee: University of Medicine & Dentistry of New Jersey

(21) Appl. No.: 10/366,990

(22) Filed: Feb. 13, 2003

Related U.S. Application Data
(63) Continuation of application No. 09/853,121, filed on May 10, 2001.

(51) Int. Cl. C12Q 1/68; G01N 33/574

(52) U.S. Cl. 435/6; 435/7.23

(57) ABSTRACT

The present invention pertains to a monoclonal antibody, or fragment thereof, having an antigen-binding specific region for NKX3.1 and to a hybridoma cell line for producing the monoclonal antibody. The present invention also pertains to a method for detecting the presence of NKX3.1 in a sample. The method comprises (a) contacting a biopsy tissue sample with a monoclonal antibody, or a fragment thereof, having an antigen-binding specific region for NKX3.1, under conditions permitting immunospecific binding between the monoclonal antibody, or a fragment thereof, and NKX3.1 in the sample; and (b) detecting whether immunospecific binding has occurred to detect the presence of NKX3.1 in the sample.

### PRIORITY CLAIM

This application is a continuation of U.S. pat. app. Ser. No. 09/853,121, filed May 10, 2001.

### BACKGROUND OF THE INVENTION

1. Field of the Invention

The present invention pertains to a monoclonal antibody, or fragment thereof, having an antigen-binding specific region for NKx3.1 and to a hybridoma cell line for producing the monclonal antibody. The present invention also pertains to a method for detecting the presence of NKx3.1 in a sample. The method comprises (a) contacting a biopsy tissue sample with monoclonal antibody, or a fragment thereof, having an antigen-binding specific region of NKx3.1, under conditions permitting immunospecific binding between the monoclonal antibody, or a fragment thereof, and NKx3.1 in the sample and (b) detecting whether immunospecific binding has occurred to detect the presence of Nkx3.1 in the sample.

2. Description of the Background

The disclosures referred to herein to illustrate the background of the invention and to provide additional detail with respect to its practice are incorporated herein by reference and, for convenience, are referenced in the following text and respectively grouped in the appended bibliography.

Deciphering the molecular mechanisms of prostate carcinogenesis has been considerably more challenging than comparable analyses for many other epithelial carcinomas, due in part to the characteristic heterogeneity and multifocality of human prostate carcinoma, as well as the lack of suitable animal models ¹. Notably, few tumor suppresser genes have been shown definitively to be lost during prostate cancer progression, and as a consequence a molecular pathway for prostate carcinogenesis remains elusive.

Nonetheless, progress has been made in identifying chromosomal alterations that are associated with progression of prostate cancer from precursor lesions (termed prostatic intraepithelial neoplasia (PIN)) to local invasive carcinoma and ultimately metastatic disease ^{1,2}. Among these, allelic imbalance of 8p21 is particularly frequent, occurring in approximately 80% of prostate tumors, and represents an early event in prostate carcinogenesis, since it is observed in PIN as well as local invasive disease^{3, 4}. In addition, allelic imbalance of 10q23 occurs in approximately 60% of carcinomas and is associated with more advanced disease^{3,5}.

One of the candidate tumor suppressers localized to chromosomal region 8p21 is the homeobox gene NKX3.1^{6,7}, a prostate-specific regulatory gene. In particular, mouse Nkx3.1 represents the earliest known marker of prostate formation, is expressed at all stages of prostate development, and is required for normal prostatic ductal morphogenesis and secretory function ⁸⁻¹¹. Furthermore, loss of Nkx3.1 function results in prostatic epithelial hyperplasia and dysplasia in mutant mice ⁹. However, despite these observations in mice, the role of NKX3.1 in human prostate carcinogenesis has been unclear, due to the lack of NKX3.1 mutations in cancer specimens ⁷.

A leading candidate tumor suppresser gene in chromosomal region 10q23 is PTEN, which represents one of the most frequently mutated genes in human cancers ¹². PTEN encodes a lipid phosphatase that functions as a negative regulator of phosphatidylinositol (3,4,5)-triphosphate (PIP-3) signaling ^{13,14} and, thereby, an inhibitor of the serine/threonine kinase Akt ¹⁵⁻¹⁷. Although Pten homozygous mice are embryonic lethal, Pten heterozygotes develop epithelial hyperplasia and dysplasia of multiple tissues, including the prostate ¹⁸⁻²⁰. However, as is the case for many other tumor suppresser genes, the mutational status of PTEN in human prostate cancer remains unresolved ²¹⁻²³).

We have been utilizing a candidate gene approach in mutant mouse models to assemble a molecular pathway for prostate carcinogenesis. Here, we report that Nkx3.1 is a tumor suppresser gene whose loss-of-function in mutant mice models prostate cancer initiation in humans, and that loss of Nkx3.1 collaborates with loss of Pten in cancer progression. Additionally, these results suggest that the biochemical mechanism for Nkx3.1 and Pten cooperatively involves their independent activation of Akt (protein kinase B), a key regulator of cellular proliferation and survival.

### SUMMARY OF THE INVENTION

The present invention pertains to a monoclonal antibody, or fragment thereof, having an antigen-binding specific region for NKX3.1 and to a hybridoma cell line for producing the monoclonal antibody. The present invention also pertains to a method for detecting the presence of NKx3.1 in a sample. The method comprises (a) contacting a biopsy tissue sample with a monoclonal antibody, or a fragment thereof, having an antigen-binding specific region for NKx3.1, under conditions permitting immunospecific binding between monoclonal antibody, or a fragment thereof, and NKx3.1 in a sample; and (b) detecting whether immunospecific binding has occurred to detect the presence of NKx3.1 in the sample.

### BRIEF DESCRIPTION OF THE FIGURES

FIG. 1 illustrates the tumor suppressor activities of Nkx3.1. FIG. 1(A) is a Western blot analysis showing expression of Nkx3.1 or Nkx3.1(L-S) proteins (arrow) following retroviral gene transfer of PC3 and AT6 cells. FIG. 1(B) illustrates cellular proliferation assays performed with AT6 or PC3 cells infected with a control retrovirus (Vector) or retroviruses expressing Nkx3.1 or Nkx3.1(L-S). Assays were performed in triplicate; error bars represent one standard deviation. FIGS. 1(C) and 1(D) illustrate anchorage-independent growth assays performed following retroviral infection of AT6 cells. Representative soft agar plates are shown in FIG. 1(C) and quantitation of assays performed in triplicate are shown in FIG. 1(D); error bars represent one standard deviation. FIG. 1(E) illustrates tumor growth in nude mice following injection of retrovirally-infected AT6 or PC3 cells. In the box plot, the horizontal line within the box represents the median tumor weight, the box represents one standard deviation, the vertical lines show two standard deviations, and the circles are outliners.

FIG. 2 illustrates loss of NKX3.1 protein expression in human prostate cancer, with immunohistochemical analysis of NKX3.1 protein expression in formalin-fixed prostatectomy specimens. FIGS. 2(A-C) illustrate examples of NKX3.1 immunostaining of normal prostate epithelium in FIGS. (2A-B) and BPH in FIG. 2(C). Note absence of staining in the basal cells (arrows) and adjacent stroma. Inset: High power view of nuclear staining of secretory epithelial cells (arrow). FIGS. 2(D-I) illustrate examples of NKX3.1 immunostaining of PIN and carcinoma. FIG. 2(D) illustrates a low power view showing staining in PIN and graded reduction of staining in the adjacent, poorly differentiated cancer. FIGS. 2(E,F) illustrate low and high power views showing low level staining in well-differentiated cancer. Note the distinct levels of staining in the same and adjacent ducts (arrows). Inset: Absence of nuclear staining in cancer cells (arrow). FIG. 2(G) illustrates a high power view showing low level staining in a heterogeneous region of moderately and poorly differentiated cancer. Note the diffuse cytoplasmic staining in the cancer duct (top arrow), contrasting with the nuclear staining of the adjacent relatively normal ducts (bottom arrow). FIG. 2(H) illustrates reduced staining in PIN and adjacent well-differentiated cancer, with higher staining intensity in PIN relative to the adjacent carcinoma. FIG. 2(I) illustrates predominantly cytoplasmic staining of NKX3.1 in poorly differentiated cancer (arrows). Inset: High power view of cytoplasmic staining. Abbreviations: NPE, normal prostate epithelium; BPH, benign prostatic hyperplasia; CaP, prostate cancer; PIN, prostatic intraepithelial neoplasia. Scale bars represent 100 microns.

FIG. 3 illustrates the Nkx3.1 mutant mice model of prostate cancer initiation. FIGS. 3(A-H) illustrate hematoxylin-eosin staining of paraffin sections of anterior prostate in wild-type (Nkx3.1^{+/+}) and homozygous (Nkx3.1^{-/-}) mice at 19 months of age. FIGS. 3(A-D) illustrate low and high power views of Nkx3.1^{+/+} prostate showing well-differentiated columnar epithelial cells arranged in papillary tufts (arrows in A); basal cells are evident (arrows in C, D) and luminal spaces are filled with secretions (lightly staining eosinophilic material). FIGS. 3 (E-H) illustrate multi-layered hyperplastic and severely dysplastic epithelium of Nkx3.1^{-/-} prostate (arrows), with little luminal space or secretory material. The insets show nuclear atypia with prominent and multiple nucleoli. FIGS. 3(I-L) illustrate immunohistochemical analysis of formalin-fixed sections of Nkx3.1^{+/+} and Nkx3.1^{-/-} anterior prostates at 12 months of age. FIGS. 3 (I, J) illustrate immunodetection of basal epithelium with anti-cytokeratin 14 antibody (CK14), showing the intact basal layer in the Nkx3.1^{+/+} prostate (I, arrows and inset). In contrast, there are disorganized basal cells at the margins of the PIN regions of the Nkx3.1^{-/-} prostate (J, arrows and inset) while the interior lacks basal cells. FIGS. 3(K L) illustrate immunodetection of smooth muscle stroma with an anti-actin antisera and show reduction of the fibromuscular sheath, and thus an increased epithelial:stromal ratio, in the Nkx3.1^{-/-} prostate relative to the Nkx3.1^{+/+} prostate. Scale bars represent 100 microns.

FIG. 4 illustrates that the loss of Nkx3.1 and Pten cooperate in prostate carcinogenesis. Hematoxylin-eosin staining of paraffin sections of anterior prostate of Nkx3.1;Pten compound mutant mice at 6 months of age. FIGS. 4(A, B) illustrate well-differentiated columnar epithelium of the Nkx3.1^{+/+};Pten^{+/+} prostate. Inset: High power views of columnar epithelial and basal cells. FIGS. 4(C, D) illustrate focal regions of dysplastic cells (arrows) surrounded by well-differentiated epithelium of the Nkx3.1^{+/+};Pten^{+/-} prostate. Inset: Example of nuclear atypia. FIGS. 4(E, F) illustrate foci of moderately hyperplastic epithelium of the Nkx3.1^{+/-};Pten^{+/+} prostate. FIGS. 4(G, H) illustrate a focal lesion of ductal carcinoma in situ (arrow) surrounded by well-differentiated epithelium of the Nkx3.1^{+/-};Pten^{+/-} prostate. FIGS. 4(I, J) illustrate extensively hyperplastic and dysplastic epithelium of the Nkx3.1^{-/-};Pten^{+/+} prostate. Inset: High power view shows example of nuclear atypia. FIGS. 4(K, L) illustrate large focal lesions of ductal carcinoma in situ surrounded by well-differentiated epithelium of the Nkx3.1^{-/-};Pten^{+/-} prostate. Inset: High power view shows atypical nuclei with a mitotic figure. Scale bars represent 100 microns.

FIG. 5 illustrates immunohistochemical analysis of prostatic lesions of Nkx3.1;Pten compound mutants. FIGS. 5(A-D) illustrates whole mounts of anterior prostates from Nkx3.1;Pten compound mutants at 6 months showing light-dense masses corresponding to ductal carcinoma in situ lesions (arrows). Bright field (A, B) and dark field (C, D) images are shown. Scale bars represent 500 microns. FIGS. 5(E-P) illustrates immunohistochemical analysis of formalin-fixed sections of the anterior prostate of Nkx3.1;Pten compound mutants at 6 months of age. FIGS. 5(E, F) illustrate immunodetection of wide spectrum cytokeratins (polycytokeratin; CK-P), which stains the membrane of normal prostate epithelium (arrow). Note the high level staining in ductal carcinoma in situ lesions of the Nkx3.1^{+/-};Pten^{+/-} prostate, indicating cytoskeletal reorganization. FIGS. 5(G, H) illustrate immunodetection of basal cells with CK14, which stains the periphery of the carcinoma in situ lesions of the Nkx3.1^{-/-};Pten^{+/-} prostate. FIGS. 5(I, J) illustrate immunodetection of endothelial cells with CD105 (endoglin) showing increased microvascularization (arrows) of the carcinoma in situ lesions of the Nkx3.1^{+/-};Pten^{+/-} prostate. FIGS. 5(K, L) illustrate that immunodetection with KI67 antibody shows increased proliferative index in the carcinoma in situ lesions (arrows indicate positive cells). In FIGS. 5(M-P), immunodetection with anti-mouse Nkx3.1 antisera (Nkx3.1) shows absence of Nkx3.1 staining in the carcinoma in situ lesions (arrows), contrasting with the robust nuclear staining of flanking, unaffected regions. Arrow in (P) shows a mitotic figure in the lesion. Scale bars represent 100 microns.

FIG. 6 illustrates the mechanism of Nkx3.1 and Pten cooperativity. FIGS. 6(A, B) illustrates a Southern blot analysis of genomic DNA recovered by laser capture microdissection of Nkx3.1 immunostained sections of ductal carcinoma in situ lesions from Nkx3.1^{+/-};Pten^{+/-} prostates. Genomic DNA from a total of 20 independent lesions that lacked Nkx3.1 staining were analyzed; representative data from 6. lesions (1-6) are shown. Control DNA (labeled with Nkx3.1) were recovered from flanking regions that retained Nkx3.1 staining. Note that the Nkx3.1 wild-type allele is retained in each case, whereas the Pten wild-type allele is lost (LOH) in all but one case (#2). FIGS. 6(C-H) illustrates immunohistochemical analysis of phospho-Akt staining of the anterior prostates from Nkx3.1;Pten compound mutants at 6 months of age (FIGS. 6 C, D), or Nkx3.1^{-/-} single mutants at 13 months (FIG. 6E), 8 months (FIG. 6F) or 26 months of age (FIGS. 6G and 6H). FIG. 6(C) illustrates a low power view showing absence of staining in the wild-type prostate. FIG. 6(D) illustrates robust staining in the ductal carcinoma in situ lesions of the Nkx3.1^{-/-};Pten^{+/-} prostate. FIG. 6(E) illustrates an example of membrane staining for phospho-Akt in a Nkx3.1^{-/-} prostate. FIGS. 6(F-H) illustrate examples of Nkx3.1^{-/-} prostates with clusters of cells showing nuclear phospho-Akt staining. Inset: High power view of cell clusters with nuclear staining. FIG. 6(I) illustrates a model for the biochemical basis of Nkx3.1 and Pten cooperativity involves their ability to independently regulate Akt activation.

### DETAILED DESCRIPTION OF THE INVENTION

The generation of mutant mouse models for investigating oncogenic progression is particularly valuable for understanding human prostate cancer, since little is known about the molecular mechanisms underlying this disease. Here, we show that loss of the homeobox gene NKx3.1 and the lipid phosphatase Pten represent critical steps in a pathway of prostate carcinogenesis, and the corresponding mutant mice model human prostate cancer. First, we find that NKx3.1 is a prostate-specific tumor suppressor gene, and that loss-of-function mutant mice display histopathological defects characteristic of prostate cancer initiation in humans. Secondly, NKx3.1 cooperates with Pten in prostate cancer progression, based on the accelerated formation of lesions resembling ductal carcinoma in situ in compound mutant mice. Thirdly, inactivation of NKx3.1 occurs through loss of protein expression in these mouse lesions as well as in human prostate cancer specimens. Finally, we present evidence that the biochemical mechanism for NKx3.1 and Pten cooperativity involves their independent activation of Akt (protein kinase B), a key regulator of cell growth and survival. We propose that interactions between tissue-specific regulators and broad-spectrum tumor suppressors underlie the distinct phenotypes of different cancers.

### RESULTS

### Tumor Suppressor Activities of Nkx3.1

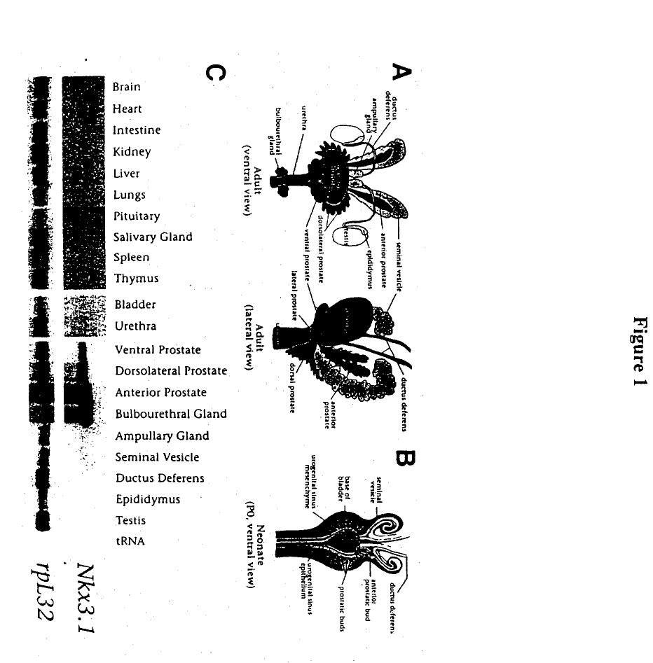

Since the ability of Nkx3.1 to function as a tumor suppressor gene has not been previously evaluated, we assessed its effects on growth and tumorigenicity of prostate carcinoma cell lines. To misexpress Nkx3.1, we employed retroviral gene transfer using a derivative of pLZRS ²⁴ that contains IRES-GFP sequences, and enriched for GFP-expressing cells by flow cytometry. Following cell sorting, greater than 95% of the cells expressed GFP as well as high levels of Nkx3.1 protein (FIG. 1A and data not shown). We compared the activity of Nkx3.1 to that of a mutated derivative, Nkx3.1(L-S), containing a substitution of a conserved residue in the homeodomain. The resulting mutant protein is stable and localizes to the nucleus (as does wild-type Nkx3.1), but is inactive in DNA-binding and transcription assays (P. Sciavolino and C. A.-S., unpublished observations).

We examined the consequences of Nkx3.1 misexpression using human (PC3) and rodent (AT6) prostate carcinoma cell lines that do not express endogenous Nkx3.1 (FIG. 1A) ^{25,26}. These results showed that misexpression of Nkx3.1, but not Nkx3.1(L-S), resulted in a 73% reduction in cellular proliferation in AT6 cells and a 59% reduction in PC3 cells (FIG. 1B). We also found that misexpression of Nkx3.1 resulted in 58% reduction in anchorage-independent growth of AT6 cells (p<0.05) (FIGS. 1C,D). Moreover, Nkx3.1-expressing AT6 and PC3 cells displayed decreased tumor growth in nude mice of 47% or 59%, respectively (p<0.01) (FIG. 1E). Similar results were obtained in all assays using a human NKX3.1 retrovirus, as well as stable tetracycline-inducible cell lines expressing mouse or human NKX3.1 (data not shown). These tumor suppressor activities of Nkx3.1 in cell culture and nude mice are consistent with the observation that Nkx3.1 mutant mice display increased proliferation of prostatic epithelium ⁹.

### Loss of NKX3.1 Protein in Human PIN and Prostate Cancer

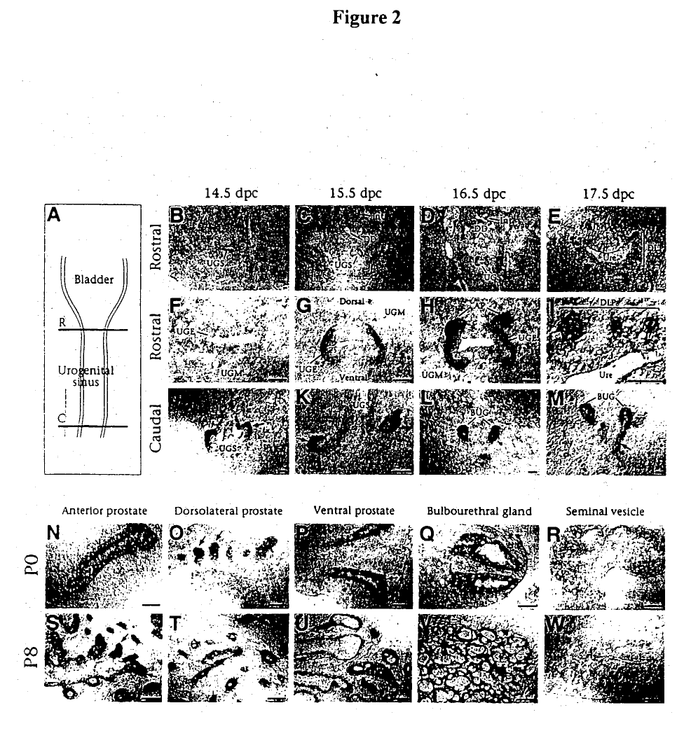

Despite these activities of NKX3.1 and its localization to 8p21, previous studies have failed to detect mutational inactivation of the coding sequence in human prostate carcinoma ⁷; we have confirmed these findings by direct sequence analysis of genomic DNA from prostate tumors (data not shown). Therefore, we have investigated NKX3.1 protein expression by immunohistochemistry, which has revealed a significant reduction in its expression in PIN as well as cancer (FIG. 2; Table 1).

In normal prostate epithelium and benign prostatic hyperplasia (BPH), NKX3.1 immunostaining was robust in the nuclei of luminal epithelial cells, but was absent in the underlying basal epithelium or adjacent stroma (FIGS. 2A-C). In contrast, NKX3.1 expression was significantly reduced (56%; n=15/27) or lost (26%; n=7/27) in a majority of prostate cancers (FIGS. 2D-I; Table 1); a similar conclusion was obtained by Gelmann and colleagues using tissue microarrays ²⁷. Notably, NKX3.1 protein expression was also reduced (58%; n=14/24) or lost (17%; n=4/24) in PIN (FIGS. 2D,H; Table 1). Interestingly, the level of NKX3.1 expression in PIN generally paralleled that in adjacent regions of carcinoma (e.g., FIGS. 2D,H), consistent with the presumed precursor relationship of PIN to carcinoma (reviewed in ^{1,2}). The observed loss of NKX3.1 protein expression at early stages of prostate carcinogenesis is consistent with a functional role for NKX3.1 inactivation during prostate cancer initiation.

One intriguing finding that we frequently observed in cancer and PIN, but never in benign tissues, was a shift in sub-cellular localization of NKX3.1 protein from nuclear to cytoplasmic (66%; n=18/27) (e.g., FIGS. 2G,I). Since NKX3.1 is a putative transcription factor that is presumed to function in the nucleus ⁸, these data suggest that NKX3.1 inactivation may sometimes occur through aberrant sub-cellular localization.

### Loss of Function of Nkx3.1 in Mutant Mice Models Prostate Cancer Initiation

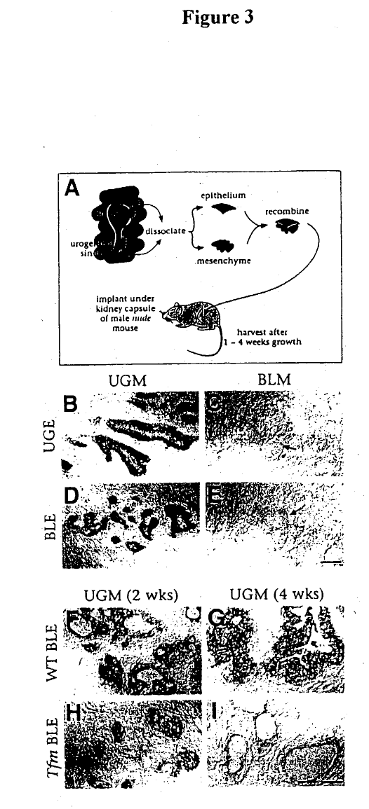

Previously, we showed that homozygous and heterozygous Nkx3.1 mutants develop prostatic epithelial hyperplasia and dysplasia prior to one year of age ⁹. We have now found that Nkx3.1 mutant mice are highly prone to develop PIN (FIG. 3; Table 2), supporting a functional role for Nkx3.1 in prostate cancer initiation. In particular, in Nkx3.1 mutants approaching 2 years of age, a majority of homozygotes (61%; n=22/36) and an intermediate number of heterozygotes (23%; n=7/30) develop histological features that define human PIN, including cribriform or papillary architecture, atypical nuclei, and enlarged nucleoli (FIGS. 3A-H). These PIN regions in Nkx3.1 prostates display additional histopathological alterations that characterize human PIN and cancer (FIGS. 3I-L), including loss of the basal layer of the epithelium as well as increased epithelial-stroma ratio, which likely reflect a decreased dependence of the secretory epithelium on the supporting basal cells and stroma.

In particular, in wild-type mice, the basal epithelium forms a discontinuous layer underlying the secretory luminal cells, while in Nkx3.1 mutants the basal layer is lost within the regions of PIN (FIGS. 3I,J). In addition, the stromal layer, comprised mainly of smooth muscle, is significantly reduced in size in Nkx3.1 mutants relative to wild-type, indicative of an increased epithelial-stromal ratio (FIGS. 3K,L). In contrast, Nkx3.1 mutant mice display no increase in neuroendocrine cells, as assessed by staining with anti-chromogranin A antisera (data not shown); such neuroendocrine cells represent a small sub-population of epithelial cells that are often amplified in advanced prostate carcinoma, but rarely in PIN. Thus, Nkx3.1 mutant mice model key histopathological features of early stages of human prostate carcinogenesis.

### Nkx3.1 and Pten Cooperate in Prostate Cancer Progression

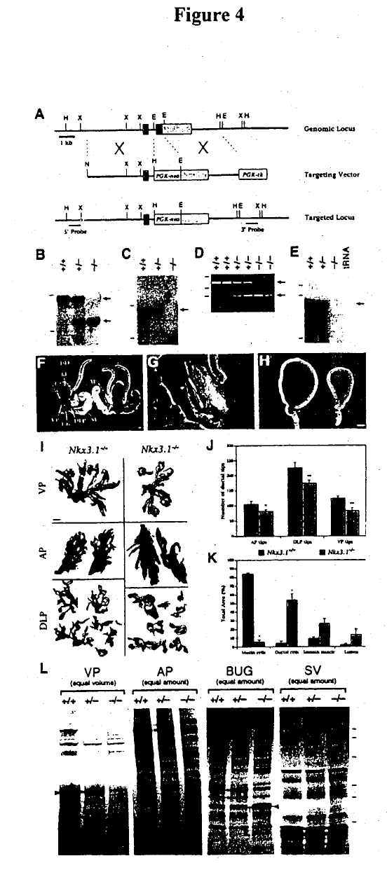

Although Pten heterozygous mice also develop prostatic epithelial hyperplasia and dysplasia ¹⁸⁻²⁰, we have observed several striking differences between the histological phenotypes of Pten and Nkx3.1 mutant prostates (FIG. 4). Overall, the histology of the Pten^{+/-} prostates was relatively normal, but displayed limited focal regions of dysplastic epithelium (FIGS. 4C,D). In contrast, the histology of the Nkx3.1 mutants displayed more broadly hyperplastic and dysplastic epithelium (FIGS. 4E,F,I,J). Moreover, while the Nkx3.1 phenotype is more prominent in the anterior prostatic lobe ⁹, the Pten phenotype is similar in the anterior and dorsolateral lobes (data not shown).

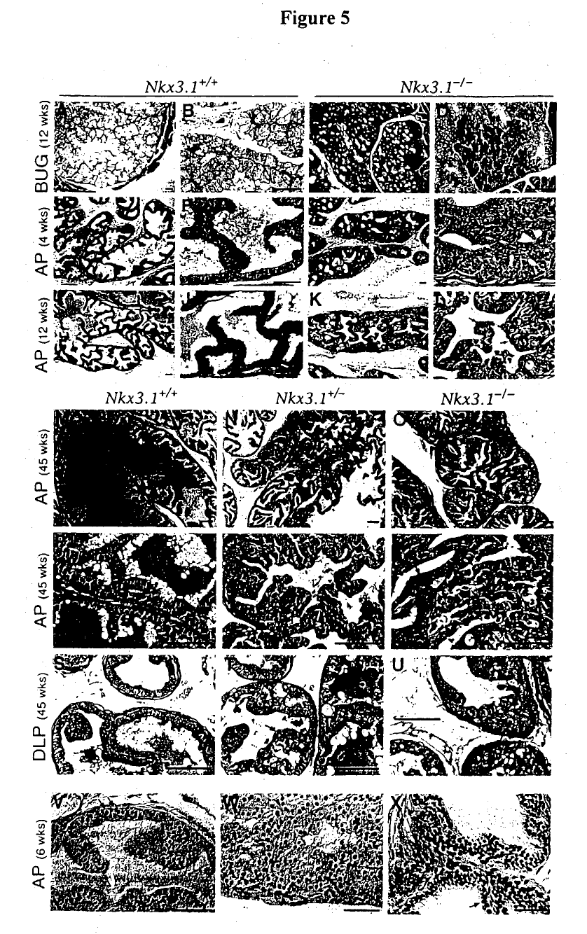

To examine whether Nkx3.1 collaborates with Pten in prostate carcinogenesis, we intercrossed compound heterozygotes (Nkx3.1^{+/-};Pten^{+/-}) to produce cohort groups comprised of all six viable genotypes. Since Pten heterozygotes generally succumb to lymphomas and other tumors by one year of age ¹⁸⁻²⁰, we analyzed Nkx3.1;Pten cohort groups from 5 to 8 months of age. These results show a striking cooperativity between Nkx3.1 and Pten that leads to formation of lesions that resemble prostatic ductal carcinoma in situ in Nkx3.1^{+/-};Pten^{+/-} and Nkx3.1^{-/-};Pten^{+/-} mice (FIGS. 4, 5; Table 3).

In particular, Nkx3.1^{+/-};Pten^{+/-} and Nkx3.1^{-/-};Pten^{+/-} compound mutant mice developed large focal lesions comprised of poorly differentiated cells with prominent and multiple nucleoli, increased nuclear:cytoplasmic ratio, and frequent mitotic figures (FIGS. 4G,H,K,L). These lesions usually filled the affected prostatic ducts, often appearing to spread within the ductal network, and were highly vascularized (FIGS. 5I,J). Based on their undifferentiated cytology, microvascularization, and high proliferative index, we define these lesions as prostatic ductal carcinoma in situ. Notably, these lesions were larger and more prevalent in the Nkx3.1^{-/-};Pten^{+/-} mice as compared with the Nkx3.1^{+/-};Pten^{+/-} mice at 6 months of age (Table 3). Similar, but significantly smaller, lesions were only occasionally seen in aged-matched Pten^{+/-} mice (Table 3), although they became more common in Pten^{+/-} mice at one year of age (data not shown).

Strikingly, these carcinoma in situ lesions are readily discernible as light-dense regions within the intact prostatic ducts, which are normally transparent (FIGS. 5A-D). Their histopathological features include a marked elevation and altered subcellular distribution of wide spectrum cytokeratins (FIGS. 5E,F), and an absence of basal epithelium (FIGS. 5G,H). In addition, the lesions display a high proliferative index, as indicated by the prevalence of mitotic figures and the abundance of Ki67-labeled nuclei (15%) (FIGS. 4L, 5K,L,P). Interestingly, outside the lesions, the Nkx3.1;Pten compound mutants do not display an increased proliferative index relative to Nkx3.1 single mutants, suggesting that Pten heterozygosity does not significantly affect cellular proliferation of the prostatic epithelium.

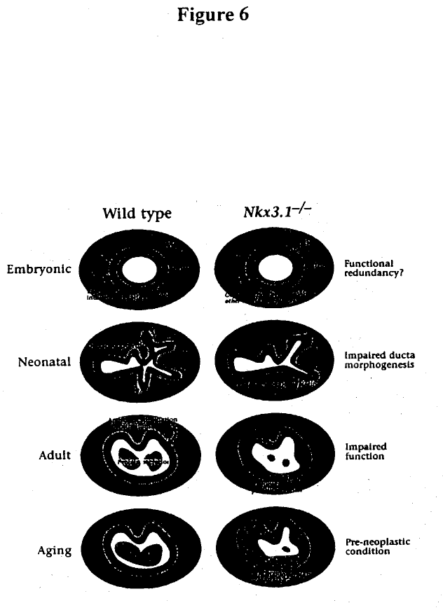

Notably, immunohistochemical analysis revealed a loss of Nkx3.1 protein expression within the carcinoma in situ lesions of Nkx3.1^{+/-};Pten^{+/-} compound heterozygotes, contrasting with its robust nuclear staining in the adjacent, unaffected regions (FIGS. 5M-P). Moreover, although similar lesions are infrequent in the Pten^{+/-} single mutant mice, they also displayed loss of Nkx3.1 protein expression (FIG. 5N). We asked whether the loss of Nkx3.1 protein expression was a consequence of the loss of the Nkx3.1 wild-type allele (loss of heterozygosity, LOH), using genomic DNA recovered by laser-capture microdissection of Nkx3.1-immunostained sections (FIG. 6A). In all cases analyzed (n=20 non-Nkx3.1 expressing regions and 8 flanking Nkx3.1-expressing controls), the wild-type Nkx3.1 allele was retained despite the absence of Nkx3.1 protein expression. In contrast, Pten sustained allelic loss (LOH) in 9 out of 10 carcinoma in situ lesions (FIG. 6B). These findings in Nkx3.1^{+/-};Pten^{+/-} mice are strikingly reminiscent of the loss of NKX3.1 protein expression in human prostate tumors that occurs without mutation of the corresponding gene, suggesting that inactivation of Nkx3.1 by loss of protein expression represents a common mechanism in mouse and human prostate carcinogenesis.

### Mechanism of Nkx3.1 and Pten Cooperativity

Finally, we examined the biochemical mechanism for the observed cooperativity between Nkx3.1 and Pten by investigating whether these genes affect a common signaling pathway. Since Pten functions as a negative regulator of PIP-3 synthesis, and thereby of activation of the Akt kinase (¹⁵⁻¹⁷ and reviewed in ¹²), we examined the status of Akt activation in Nkx3.1 and Pten single mutants and Nkx3.1;Pten compound mutant prostates using an antibody that detects the activated (phosphorylated) kinase (FIGS. 6C-H). Consistent with loss of Pten activity, we observed that Akt was highly activated in the ductal carcinoma in situ lesions. Notably, however, we also observed Akt activation in Nkx3.1 mutant prostates, suggesting that loss of Nkx3.1 independently affects Akt signaling.

In particular, we observed that phospho-Akt staining was undetectable in unaffected regions of prostatic epithelium in the Pten^{+/-} single mutants as well as the Nkx3.1;Pten compound mutants (FIGS. 6C,D and data not shown). In contrast, we found that phospho-Akt staining was highly elevated in carcinoma in situ lesions occurring in these mice, where it was primarily localized to the cell membrane (FIG. 6D).

Notably, we also observed Akt activation in Nkx3.1 single mutant prostates (n=8) (FIGS. 6E-H). In some cases, activated Akt was localized to the membrane, similar to that observed in the ductal carcinoma in situ lesions of Nkx3.1;Pten compound mutants (FIG. 6E). More commonly, however, we observed nuclear localization of activated Akt in isolated small groups of prostatic epithelial cells, which were generally correlated with the presence of PIN lesions and found near ductal tips (FIGS. 6F-H). The nuclear localization of activated Akt in Nkx3.1 mutants is noteworthy since this kinase is believed to function in the nucleus as well as the cytoplasm, and has been implicated in phosphorylating nuclear targets ²⁸⁻³⁰. No positive staining was observed in wild-type control prostates, or in other epithelial tissues from Nkx3.1 mutants such as bladder and intestine, where Nkx3.1 is not expressed (FIG. 6C and data not shown). These findings suggest that the observed cooperativity of Nkx3.1 and Pten in prostate carcinogenesis is due to their ability to affect Akt activation by independent pathways (FIG. 6I), and underscore a novel role for Nkx3.1 in regulation of Akt signaling.

### DISCUSSION

Until recently, the validity of the mouse as a model for human prostate cancer has been questionable, due to the anatomical and histological differences between mouse and human prostate and the absence of spontaneous prostate cancer in the mouse (reviewed in ¹). Here, we have developed mutant mouse models that accurately recapitulate early stages of human prostate carcinogenesis and provide novel mechanistic insights into these processes. These analyses represent a significant step toward utilizing mouse models to assemble a molecular pathway for human prostate cancer progression.

These findings establish a role for loss of NKX3.1 function in prostate cancer initiation in the mouse and provide strong support for a corresponding role in human cancer. Indeed, these functional analyses implicate NKX3.1 as an excellent candidate for the tumor suppressor activity at the 8p21 locus, and are consistent with allelotyping studies of 8p that have defined a minimal gene deletion interval of 500 kb containing the NKX3.1 locus (M. Emmert-Buck, personal communication). However, NKX3.1 does not represent a classical tumor suppressor gene, since it does not undergo mutational inactivation either in human prostate cancer or in mouse models. Instead, NKX3.1 inactivation in both humans and mice involves loss of protein expression. Although the mechanism of protein loss is presently unknown, it is likely to involve post-transcriptional regulation, since the unusually long NKX3.1 3′UTR contains putative translational control elements that are conserved between mouse and human ⁸. Inactivation of tumor suppressor function through loss of protein expression has also been described for the cyclin-dependent kinase inhibitor p27 and for the catalytic subunit of PI(3)Kg in epithelial carcinomas ^{31,32}. Thus, these findings further expand the mechanisms of tumor suppressor gene inactivation from a classical “two-hit” model to include additional scenarios for functional inactivation.

In contrast to the prostate-specificity of NKX3.1, PTEN is a broad-spectrum and “classic” tumor suppressor gene whose loss has been implicated in many cancers, including glioblastoma as well as endometrial and breast carcinoma ¹². Despite their differences, Nkx3.1 and Pten collaborate in prostate carcinogenesis in mutant mice, and the mechanism for their synergy is likely to involve independent modes of activating Akt (FIG. 6I), which in turn is a key regulator of cellular proliferation and survival. While the mechanism by which loss of Nkx3.1 results in Akt activation is presently unknown, it is likely to be indirect, since Akt is not uniformly activated in the prostatic epithelium of Nkx3.1 mutants. Nonetheless, the convergence of the Nkx3.1 and Pten mutant phenotypes on Akt activation in the prostate also implies that de-regulation of Akt activity is a critical event in prostate cancer initiation.

Thus, we have shown that collaboration between a tissue-specific modulator of prostatic epithelial differentiation and a broad-spectrum tumor suppressor gene can result in cancer progression. We propose that such interactions contribute to the distinguishing features of prostate carcinoma relative to other cancers, and that similar interactions may explain the tissue-specific phenotypes of cancers.

The present invention is further illustrated by the following examples which are not intended to limit the effective scope of the claims. All parts and percentages in the examples and throughout the specification and claims are by weight of the final composition unless otherwise specified.

### EXPERIMENTAL PROCEDURES

### Retroviral Gene Transfer and Tumorigenicity Assays

To generate mammalian retroviruses, sequences corresponding to the coding region of mouse or human Nkx3.1 ^{6,8} or mouse Nkx3.1(L-S) were subcloned into pLZRSD-IRES-GFP, a derivative of LZRSpBMN-Z ²⁴ in which the lacZ gene was replaced with an IRES-GFP cassette. The mutant Nkx3.1(L-S) gene contains a substitution of leucine 140 to serine (homeodomain position 16), which was introduced by PCR mutagenesis. Replication-defective mammalian retroviruses were made in Phoenix amphitropic retroviral packaging cells (ATCC). Target cells were seeded at a density of 1×10⁴/cm² for PC3 cells and 5×10³/cm² for AT6 (AT6.3) cells, and infected with viral supernatants (containing 8 μg/ml polybrene) on three consecutive days. Expression of Nkx3.1 or Nkx3.1(L-S) was verified by Western blot analysis directly following flow cytometry, and also at the termination of each assay.

For proliferation assays, PC3 cells were seeded in triplicate at a density of 5×10⁴ cells/6 well dish in media containing 0.5% FBS, and AT6 cells were seeded at 1×10⁴ cells/6 well dish in media containing 0.25% FBS; media was replenished every second day. Cell number was determined by optical density following staining with Napthol blue black (Sigma). Anchorage-independent growth was monitored by seeding AT6 cells in triplicate at a density of 1,000 cells/6 well dish in media containing 0.35% agarose layered over 0.5% agar; cells were grown for 14 days. Tumor growth in nude mice (Taconic) was monitored by subcutaneous injection of AT6 cells (1×10⁴) or PC3 cells (1×10⁶ in 50% matrigel). Tumor size was monitored for four weeks (AT6) or six weeks (PC3) by measuring with calipers in two dimensions, following by determination of tumors weights at necropsy. Expression of Nkx3.1 in the tumors was verified by immunohistochemistry. Statistical analyses were performed using a two-sample t test for independent samples with unequal variances (Satterthwaite's method).

### Mutant Mouse Strains and Analyses

The Nkx3.1 and Pten mutant mice have been described ^{9,19}. Analyses were performed on a hybrid 129/SvImJ and C57B1/6J strain background using virgin male mice from postnatal day 0 through 24 months of age. For histological analyses, dissected tissues were fixed in OmniFix 2000 (Aaron Medical Industries, St. Petersburg, Fla.), and processed for hematoxylin-and-eosin staining. The primary histological analysis was performed on a non-blinded basis (by R.D.C.); one of us (M.M.S.) independently reviewed the histological data on a blinded basis, reaching similar conclusions. The human prostate tumor specimens (generously supplied by Dr. Regina Gandour-Edwards) were paraffin embedded prostate tissues retrieved from the surgical pathology files at the University of California Davis Medical Center. The histological diagnosis and Gleason grade were independently verified by one of us (R.D.C.) and Dr. Gandour-Edwards.

Immunohistochemical analysis was performed on cryosections (for Akt and phospho-Akt antibodies) or formalin-fixed tissues following antigen retrieval (for all other antibodies). Antibodies were as follows: monoclonal antibody against smooth muscle actin (Sigma); monoclonal antibody against cytokeratin 14 (Biogenex); monoclonal antibody against CD105, endoglin (DAKO); polyclonal antisera against poly-cytokeratins, for wide spectrum screeining (DAKO); polyclonal antisera against Ki67 antigen (Novocastra Laboratories); polyclonal antisera against Akt and phospho-Akt (Ser 473) (Cell Signaling Technology). Anti-NKX3.1 antisera were generated using full-length mouse or human NKX3.1 proteins purified from E. coli lysates as hexa-histidine fusion proteins. The data shown in FIG. 2 were performed using anti-NKX3.1 polyclonal antisera; similar results were obtained with an anti-NKX3.1 monoclonal antibody (data not shown). Immunodetection was performed using Vector M.O.M. immunodetection kit for monoclonal antibodies or Vector Elite ABC kit Rabbit IgG for polyclonal antisera with Vector NovaRED substrate kit (Vector Laboratories). Ki67-labelled nuclei were quantitated by counting approximately 20,000 hematoxylin-stained nuclei from high-power microscopic fields.

Laser-capture microdissection was performed on immunostained sections using a PixCell apparatus (Arcturus Eng. Inc.). DNA was extracted from pooled samples (1000 laser pulses) at 37° C. in 50 mM Tris-HCl (pH 8.5), 0.5% Tween-20, 1 mM EDTA (pH 8.0), and 0.5 mg/ml Proteinase K. DNA was analyzed by PCR amplification followed by southern blot analysis. Primer sequences were as follows: For the Nkx3.1 wild type allele, 5′-GCCACAGTGGCTGATGTCAAGGAGTCGG (primer A) (SEQ ID NO: 1) and 5′-GCCAACCTGCCTCAATCACTAAGG (SEQ ID NO: 2). For the Nkx3.1 targeted allele, primer A and 5′-TTCCACATACACTTCATTCTCAGT (SEQ ID NO: 3). For the Pten wild type allele (exon 5), 5′-AAAAGTCAGTCTTTTCCATAGTTGA (primer B) (SEQ ID NO: 4) and 5′-AATATAACAGTTCTCAAAGCATCA (SEQ ID NO: 5). For the Pten targeted allele, primer B and 5′-TAGCGCCAAGTGCCCAGCGGGGC (SEQ ID NO: 6).

In another embodiment the present invention is directed to a Nkx3.1 promoter. Promoters are DNA sequences found upstream of a gene that promote transcription of a gene to produce mRNA and may be the attachment site for RNA polymerase. A Nkx3.1 promoter will direct expression specifically in the prostate. In particular, these findings have shown that Nkx3.1 is expressed early during prostate development and into adulthood. A prostate-specific promoter will be of commercial use in potential gene therapy and for other strategies to direct therapeutics to the prostate.

TABLE 1
Summary of NKX3.1 expression in human prostate tissue.

| Staining | | | | |
|---|---|---|---|---|
| intensity^{a} | Normal | BPH | PIN | Carcinoma^{b} |
| 4 | 21/27 (78%) | 22/27 (81%) | 3/24 (12.5%) | 3/27 (11%) |
| 3 | 4/27 (15%) | 3/27 (12%) | 3/24 (12.5%) | 2/27 (7%) |
| 2 | 2/27 (7%) | 2/27 (7%) | 10/24 (41%) | 11/27 (41%) |
| 1 | 0/27 (0%) | 0/27 (0%) | 4/24 (17%) | 4/27 (15%) |
| 0 | 0/27 (0%) | 0/27 (0%) | 4/24 (17%) | 7/27 (26%) |

^{a)}Staining intensity was scored using an arbitrary scale of 0 to 4, with 4 being the highest level of staining and 0 representing no staining.
^{b)}The carcinomas corresponding to Gleason grades 6-9; no significant correlation was observed between NKX3.1 protein expression and Gleason grade (data not shown).

TABLE 2
Summary of prostatic epithelial defects in the anterior prostate of
Nkx3.1 mutant mice^{a}

| Genotype | Total # | Normal | Hyperplasia | PIN |
|---|---|---|---|---|
| +/+ | | | | |
| 1-6 month | N = 11 | 11 | 0 | 0 |
| 6-12 month | N = 6 | 4 | 1 | 1 |
| 12-24 month | N = 11 | 9 | 2 | 0 |
| | N = 28 | 24 | 3 | 1 |
| +/− | | | | |
| 1-6 month | N = 12 | 9 | 3 | 0 |
| 6-12 month | N = 7 | 2 | 2 | 3 |
| 12-24 month | N = 11 | 3 | 4 | 4 |
| | N = 30 | 14 | 9 | 7 |
| −/− | | | | |
| 1-6 months | N = 13 | 2 | 5 | 6 |
| 6-12 month | N = 8 | 3 | 1 | 5 |
| 12-24 month | N = 15 | 0 | 5 | 11 |
| | N = 36 | 5 | 11 | 22 |

^{a)}Data for the mice at 1-12 months includes data previously reported in ⁹.

TABLE 3
Summary of the prostatic epithelial defects in the anterior prostate of
Nkx3.1;Pten compound mutant mice at 5-8 months of age

| | | | | | Carcinoma |
|---|---|---|---|---|---|
| Genotype | Total # | Normal | Hyperplasia | PIN | in situ |
| Nkx3.1^{+/+};Pten^{+/+} | N = 6 | 5 | 1 | 0 | 0 |
| Nkx3.1^{+/-};Pten^{+/+} | N = 11 | 6 | 4 | 1 | 0 |
| Nkx3.1^{-/-};Pten^{+/+} | N = 10 | 2 | 4 | 4 | 0 |
| Nkx3.1^{+/+};Pten^{+/-} | N = 10 | 3 | 2 | 5 | 2 |
| Nkx3.1^{+/-};Pten^{+/-} | N = 13 | 2 | 3 | 8 | 8 |
| Nkx3.1^{-/-};Pten^{+/-} | N = 11 | 0 | 2 | 9 | 11 |

### REFERENCES

1. Abate-Shen, C. & Shen, M. M. Molecular genetics of prostate cancer. Genes Dev 14, 2410-2434 (2000).

2. Bostwick, D. G. & Brawer, M. K. Prostatic intra-epithelial neoplasia and early invasion in prostate cancer. Cancer 59, 788-794 (1987).

3. Bergerheim, U. S., Kunimi, K., Collins, V. P. & Ekman, P. Deletion mapping of chromosomes 8, 10, and 16 in human prostatic carcinoma. Genes Chromosomes Cancer 3, 215-220 (1991).

4. Emmert-Buck, M. R. et al. Allelic loss o n chromosome 8p12-21 in microdissected prostatic intraepithelial neoplasia. Cancer Res 55, 2959-2962 (1995).

5. Ittmann, M. Allelic loss on chromosome 10 in prostate adenocarcinoma. Cancer Res 56, 2143-2147 (1996).

6. He, W. W. et al. A novel human prostate-specific, androgen-regulated homeobox gene (NKX3.1) that maps to 8p21, a region frequently deleted in prostate cancer. Genomics 43, 69-77 (1997).

7. Voeller, H. J. et al. Coding region of NKX3.1, a prostate-specific homeobox gene on 8p21, is not mutated in human prostate cancers. Cancer Res 57, 4455-4459 (1997).

8. Sciavolino, P. J. et al. Tissue-specific expression of murine Nkx3.1 in the male urogenital system. Dev Dyn 209, 127-138 (1997).

9. Bhatia-Gaur, R. et al. Roles for Nkx3.1 in prostate development and cancer. Genes Dev 13, 966-977 (1999).

10. Schneider, A., Brand T., Zweigerdt, R. & Arnold, H. Targeted disruption of the nkx3.1 gene in mice results in morphogenetic defects of minor salivary glands: parallels to glandular duct morphogenesis in prostate. Mech Dev 95, 163-174 (2000).

11. Tanaka, M. et al. Nkx3.1, a murine homolog of drosophila bagpipe, regulates epithelial ductal branching and proliferation of the prostate and palatine glands [In Process Citation]. Dev Dyn 219, 248-260 (2000).

12. Di Cristofano, A. & Pandolfi, P. P. The multiple roles of PTEN in tumor suppression. Cell 100, 387-390 (2000).

13. Myers, M. P. et al. The lipid phosphatase activity of PTEN is critical for its tumor supressor function. Proc Natl Acad Sci U S A 95, 13513-13518 (1998).

14. Maehama, T. & Dixon, J. E. The tumor suppressor, PTEN/MMAC1, dephosphorylates the lipid second messenger, phosphatidylinositol 3,4,5-trisphosphate. J Biol Chem 273, 13375-13378 (1998).

15. Stambolic, V. et al. Negative regulation of PKB/Akt-dependent cell survival by the tumor suppressor PTEN. Cell 95, 29-39 (1998).

16. Wu, X., Senechal, K:, Neshat, M. S., Whang, Y. E. & Sawyers, C. L. The PTEN/MMAC1 tumor suppressor phosphatase functions as a negative regulator of the phosphoinositide 3-kinase/Akt pathway. Proc Natl Acad Sci U S A 95, 15587-15591 (1998).

17. Sun, H. et al. PTEN modulates cell cycle progression and cell survival by regulating phosphatidylinositol 3,4,5,-trisphosphate and Akt/protein kinase B signaling pathway. Proc Natl Acad Sci U S A 96, 6199-6204 (1999).

18. Di Cristofano, A., Pesce, B., Cordon-Cardo, C. & Pandolfi, P. P. Pten is essential for embryonic development and tumour suppression. Nat Genet 19, 348-355 (1998).

19. Podsypanina, K. et al. Mutation of Pten/Mmac1 in mice causes neoplasia in multiple organ systems. Proc Natl Acad Sci U S A 96, 1563-1568 (1999).

20. Stambolic, V. et al. High incidence of breast and endometrial neoplasia resembling human Cowden syndrome in pten+/− mice. Cancer Res 60, 3605-3611 (2000).

21. Feilotter, H. E., Nagai, M. A., Boag, A. H., Eng, C. & Mulligan, L. M. Analysis of PTEN and the 10q23 region in primary prostate carcinomas. Oncogene 16, 1743-1748 (1998).

22. Dong, J. T. et al. PTEN/MMAC1 is infrequently mutated in pT2 and pT3 carcinomas of the prostate. Oncogene 17, 1979-1982 (1998).

23. Suzuki, H. et al. Interfocal heterogeneity of PTEN/MMAC1 gene alterations in multiple metastatic prostate cancer tissues. Cancer Res 58, 204-209 (1998).

24. Kinsella, T. M. & Nolan, G. P. Episomal vectors rapidly and stably produce high-titer recombinant retrovirus. Hum Gene Ther 7, 1405-1413 (1996).

25. Kaighn, M. E., Shanker, N., Ohnuki, Y., Lechner, J. F. & Jones, L. W. Establishment and characterization of a human prostatic carcinoma cell line (PC-3). Invest. Urol. 17, 16-23 (1979).

26. Isaacs, J. T., Isaacs, W. B., Feitz, W. F. & Scheres, J. Establishment and characterization of seven Dunning rat prostatic cancer cell lines and their use in developing methods for predicting metastatic abilities of prostatic cancers. Prostate 9, 261-281 (1986).

27. Bowen, C. et al. Loss of NKX3.1 expression in human prostate cancers correlates with tumor progression [In Process Citation]. Cancer Res 60, 6111-6115 (2000).

28. Meler, R., Alessi, D. R., Cron, P., Andjelkovic, M. & Hemmings, B. A. Mitogenic activation, phosphorylation, and nuclear translocation of protein kinase Bbeta. J Biol Chem 272, 30491-30497 (1997).

29. Andjelkovic, M. et al. Role of translocation in the activation and function of protein kinase B. J Biol Chem 272, 31515-31524 (1997).

30. Brunet, A. et al. Akt promotes cell survival by phosphorylating and inhibiting a Forkhead transcription factor. Cell 96, 857-868 (1999).

31. Tsihlias, J., Kapusta, L. & Slingerland, J. The prognostic significance of altered cyclin-dependent kinase inhibitors in human cancer. Annu Rev Med 50, 401-423 (1999).

32. Sasaki, T. et al. Colorectal carcinomas in mice lacking the catalytic subunit of PI(3)Kgamma. Nature 406, 897-902 (2000).

Throughout this disclosure, applicant will suggest various theories or mechanisms. While applicant may offer various mechanisms to explain the present invention, applicant does not wish to be bound by theory. These theories are suggested to better understand the present invention but are not intended to limit the effective scope of the claims.

While the invention has been particularly described in terms of specific embodiments, those skilled in the art will understand in view of the present disclosure that numerous variations and modifications upon the invention are now enabled, which variations and modifications are not to be regarded as a departure from the spirit and scope of the invention. Accordingly, the invention is to be broadly construed and limited only by the scope and spirit of the following claims.

1. A method for determining predisposition to prostate cancer in a patient comprising:
(a) screening for the level of tumor suppressor Nkx3.1 in urogenital tissue of the patient; and
(b) screening for the level of tumor suppressor Pten in urogenital tissue of the patient;
wherein physiologically low expression to no expression of Nkx3.1 and a physiologically low expression of Pten indicates a high risk of prostate cancer;
physiologically low expression to no expression of Nkx3.1 and normal expression Pten or physiologically low expression of Pten and normal expression of Nkx3.1 indicates a moderate risk of prostate cancer; and
physiologically normal expression of both Pten and Nkx3.1 indicates a low risk of prostate cancer.

2. The method of claim 1, wherein a physiologically low expression of Pten results from a heterozygous Pten gene and physiologically normal expression of Pten results from a homozygous positive gene.

3. The method of claim 1, wherein a physiologically low expression of Nkx3.1 results from a heterozygous Nkx3.1 gene, physiologically no expression of Nkx3.1 results from a homozygous negative Nkx3.1 gene, and physiologically normal expression of Nkx3.1 results from a homozygous positive Nkx3.1 gene.

4. The method of claim 1, wherein the screening occurs by contacting a biopsied tissue sample from the patient with tumor suppressor detecting agents.

5. The method of claim 4, wherein the tumor suppressor detecting agents are monoclonal antibodies, or fragments thereof, specific for the tumor suppressors of Nkx3.1 and Pten.

6. The method of claim 5, wherein the antibodies to Nkx3.1 and Pten are distinguishably labeled and detectable when bound to their respective tumor suppressor.

7. A method for determining predisposition to prostate cancer in a patient comprising:
(a) screening for the genotype of tumor suppressor Nkx3.1; and
(b) screening for the genotype of tumor suppressor Pten;
wherein the genotype of Nkx3.1^{+/+}; Pten^{+/+} indicates a low risk of prostate cancer, the genotypes of Nkx3.1^{+/+}; Pten^{+/-} and Nkx3.1^{+/-}; Pten^{+/+} indicate a moderate risk of prostate cancer, the genotypes of Nkx3.1^{+/-}; Pten^{+/-} and Nkx3.1^{-/-}; Pten^{+/+} indicate a high risk of prostate cancer, and the genotype of Nkx3.1^{-/-}; Pten^{+/-} indicates a very high risk of prostate cancer.

8. The method of claim 7, wherein the risk of prostate cancer is related to the independent activation of Akt by NXk3.1 and Pten.

9. The method of claim 7, wherein the genotype of Nkx3.1 is determined by using PCR to amplify the gene with nucleic acid primers SEQ ID NO: 1 and SEQ ID NO: 2 or SEQ ID NO: 3, and wherein the genotype of Pten is determined by using PCR to amplify the gene with nucleic acid primers SEQ ID NO: 4 and SEQ ID NO: 5 or SEQ ID NO: 6.
